(19) United States
(12) Patent Application Publication
(10) Pub. No.: US 2026/0090973 A1
(43) Pub. Date: Apr. 2, 2026

(54) EYE KERATIN MATERIAL COATING COMPOSITIONS

(71) Applicant: L'OREAL, PARIS (FR)

(72) Inventors: DAVID CHAN, ORADELL, NJ (US); ALLISON PERNER, METUCHEN, NJ (US)

(73) Assignee: L'OREAL, PARIS (FR)

(21) Appl. No.: 18/902,362

(22) Filed: Sep. 30, 2024

(51) Int. Cl. A61K 8/81; A61K 8/34; A61Q 1/10

(52) U.S. Cl. CPC A61K 8/8152; A61K 8/345; A61Q 1/10; A61K 2800/30; A61K 2800/54; A61K 2800/591; A61K 2800/884; A61K 2800/95

(57) ABSTRACT

The invention relates to relates to eye keratin material coating compositions including water, at least one low glass transition temperature (Tg) film-forming agent, at least one secondary alcohol compound, wherein a weighted average ratio of molecular weight/number of secondary alcohol groups for the at least one secondary alcohol compound present in the composition is less than 69, and optionally at least one nonionic amphiphilic thickening agent, as well as to methods of using such compositions and kits containing such compositions.

### FIELD OF THE INVENTION

The present invention relates to eye keratin material coating compositions comprising water, at least one low glass transition temperature (Tg) film-forming agent, at least one secondary alcohol compound, wherein a weighted average ratio of molecular weight/number of secondary alcohol groups for the at least one secondary alcohol compound present in the composition less than 69, and optionally at least one nonionic amphiphilic thickening agent, as well as to methods of using such compositions and kits containing such compositions.

### DISCUSSION OF THE BACKGROUND

Eyelash compositions such as mascaras typically contain wax which can provide body and volume to the composition. Mascaras can also contain surfactants, particularly when they are in the form of emulsions. However, mascara formulations are typically created to minimize or squelch the full effect of the properties of these ingredients, if present, in an attempt to obtain specific desired properties of the mascara being formulated such as volumization, lengthening, easy removal, etc.

Further, eyelash compositions such as mascara compositions tend to be of two types, (1) water-resistant, long-wearing mascaras which are anhydrous or (2) removable mascaras (for example, removable with soap and water) which contain water. Generally speaking, water-resistant mascaras are not easily removable, and removable mascaras are not particularly long-wearing.

Fake (artificial) eyelashes typically can be applied to natural (real) eyelashes through means such as adhesives (glue) or magnets.

For example, WO2020180346 discloses magnetically attachable eyelash prosthetic systems mediated by a magnetic mascara, US 2016/0206031 discloses the use of magnetic materials, and US 2019/0261715 discloses magnetic materials in an eyeliner-type composition.

With respect to adhesives or glue, US 2017/0027844, WO 2020/172746, and WO 2019/003454 are examples of references disclosing the use of such compounds for false (artificial) eyelashes.

U.S. Pat. No. 9,320,920 discloses compositions for making up eyelashes, including false (artificial) eyelashes.

JP6190173 discloses a false eyelash adhesive which is black, or dark color based on black in appearance after application, and is capable of using in combination with an eyeliner by imparting a quick-drying adhesive layer to an application part containing (A) 30-70 mass % of water-insoluble film-forming polymer; (B) 0.1-10 mass % of carbon black; and (C) 0.01-15 mass % of nonionic surfactant with HLB of 10 or more.

There remains a need for improved cosmetic compositions having improved cosmetic properties which can be useful for coating eye keratin materials, for example for attaching fake (artificial) eyelashes to natural (real) eyelashes, in particular such compositions which do not contain traditional glues or adhesives or magnets such as those used in prior attachment of such false (artificial) products to eye keratin materials.

Accordingly, one aspect of the present invention is a water-containing care and/or makeup and/or treatment composition for eye keratin materials which can be used to coat eye keratin materials to care for, makeup, and/or attach fake (artificial) structures (such as artificial eyelashes) to natural (real) eye keratin materials.

### SUMMARY OF THE INVENTION

The present invention relates to eye keratin material coating compositions comprising water, at least one low glass transition temperature (Tg) film-forming agent, at least one secondary alcohol compound, wherein a weighted average ratio of molecular weight/number of secondary alcohol groups for the at least one secondary alcohol compound present in the composition less than 69, and optionally at least one nonionic amphiphilic thickening agent. Preferably, the compositions are devoid of, substantially free of, or free of one or more of the following: oils, waxes, surfactants, and/or colorants. Preferably, the compositions are devoid of, substantially free of, or free of at least two of the following: oils, waxes, surfactants, and/or colorants, preferably at least three of these types of compounds, and preferably all four of these types of compounds. Also preferably, the compositions are devoid of, substantially free of, or free of hydrophilic thickening agents.

The present invention also relates to methods of caring for and/or making up real (natural) eye keratin materials by applying compositions of the present invention to natural eye keratin materials in an amount sufficient to care for and/or make up the natural eye keratin materials.

The present invention also relates to methods of applying fake (artificial) eye keratin materials such as artificial eyelashes or eyebrows to real (natural) eye keratin materials comprising applying compositions of the present invention to natural eye keratin materials, applying fake eye keratin materials to the natural eye keratin materials upon which compositions of the present invention have been previously applied, and allowing the compositions of the present invention to dry so that the fake eye keratin materials adhere or bind to the natural eye keratin materials.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Eye keratin material” as used herein refers to eye related hair such as eyelashes and eyebrows, as well as skin associated with eye related hair such as eyelids and brows.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Real” and “natural” are used interchangeably throughout this specification.

“False,” “fake” and “artificial” are used interchangeably throughout this specification.

“False,” “fake” and “artificial” “eye keratin materials” as used herein means individual fibers, clusters of fibers, or collection of fibers attached to a substrate (e.g., band) which can be attached or adhered to “eye keratin material.” The fibers may be natural, such as derived from human hair, mink or plant origin, or synthetic, such as engineered polymers such as thermoplastic polymers like polybutylene terephthalate (PBT). Examples include fibers for producing artificial eyelashes and/or artificial eyebrows.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C. “Polymer” as used herein means a compound which is made up of at least two monomers.

“Glass transition temperature (Tg)” refers to the temperature at which amorphous material changes from a glassy solid state to a rubbery state. The temperature may be measured by standard techniques in the art, such a Differential Scanning Calorimetry (DSM), e.g., according to a standard protocol such as ASTM D3418-97 standard.

“Low Tg” or “Low Glass Transition Temperature” as used herein means a glass transition temperature (Tg) of −30° C. or lower.

“Secondary alcohol compound” as used herein refers to a compound having at least one secondary alcohol group.

“Polymer” as used herein means a compound which is made up of at least two monomers, and includes homopolymers and copolymers.

Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of surfactant” means that an effective amount (that is, more than trace amounts) of surfactant is omitted from the composition (that is, about 0% by weight), “substantially free of surfactant” means that surfactant, if present, is present in amounts not greater than 1% by weight, and “devoid of surfactant” means that surfactant, if present, is present in amounts not greater than 0.5% by weight, based on the total weight of the composition.

The same nomenclature applies for all other ingredients identified throughout the application and in this paragraph such as, for example, colorants (compositions of the invention which are “free of colorants,” “substantially free of colorants,” and “devoid of colorants” have meanings consistent with the discussion within this paragraph), even if not specifically discussed for each identified ingredient. The same applies to oils and/or waxes (“free of oils and/or waxes,” “substantially free of oils and/or waxes,” and “devoid of oils and/or waxes”) and magnets (“free of magnets,” “substantially free of magnets,” and “devoid of magnets”)—such phrases should be understood to have meanings consistent with the discussion within this paragraph. Compositions of the present invention thus can be free, substantially free or devoid of any of the ingredients in this paragraph. However, discussed examples of the use of such language are intended to be exemplary, not limiting.

“Tack” as used herein refers to the quality exhibited by compositions related to initial attachment to an object after application to the object (for example, skin or eyelashes). Tack may be evaluated by any method known in the art for evaluating it, such as using a texture analyzer. For example, a sample can be applied to a substrate (for example, a 1 mil drawdown), allowed to dry (for example, for 1 minute), and contacted by an object such as a hemispherical probe (SMS P/0.5HS, ½ inch diameter cylinder hemispherical from Stable MicroSystems), after which the force associated with removal of the probe from the object can be measured and reported as tack (g). Such measurements can be performed 1 minute after the drawdown. Preferably, compositions of the present invention possess tack properties, when determined by this method, of greater than 45 g, preferably greater than 50 g, preferably greater than 70 g, preferably greater than 90 g, and preferably greater than 100 g. So, compositions of the present invention preferably have tack properties ranging from about 30 g to about 200 g, preferably from about 50 g to about 150 g, and preferably from about 70 g to about 130 g, including all ranges and subranges therebetween such as, for example, about 45 g to about 110 g, about 50 g to about 110 g, about 70 g to about 120 g, about 90 g to about 115 g, etc.

“Cohesion” as used herein refers to the quality exhibited by compositions related to resistance of the compositions after application to an object (for example, skin or eyelashes). Cohesion may be evaluated by any method known in the art for evaluating it, such as gauging the force needed to create a rupture or tear in the compositions after drying.

“Adhesion” as used herein refers to the quality exhibited by compositions related to wear of the compositions after application to an object (for example, skin or eyelashes). Adhesion may be evaluated by any method known in the art for evaluating it, such as gauging the force needed to remove the compositions from an object after application and drying.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as eye keratin materials and evaluating the color of the composition after an extended period of time. For example, the color of a composition may be evaluated immediately following application to keratin materials such as eye keratin materials and these characteristics may then be re-evaluated and compared after a certain amount of time. Further, these characteristics may be evaluated with respect to other compositions, such as commercially available compositions.

“Making up” as used herein means to provide decoration (for example, color or glitter) to the eye keratin materials.

“Protecting” as used herein means to inhibit damage to the eye keratin materials by providing a protective layer on the eye keratin materials such as, for example, through application of the compositions of the present invention, including as a basecoat and/or topcoat composition, and is encompassed within “caring for.”

The compositions and methods of the present invention can comprise, consist of, or consist essentially of the essential elements and limitations of the invention described herein, as well as any additional or optional ingredients, components, or limitations described herein or otherwise useful. For example, the glass transition temperature film forming component of the composition can “consist essentially of” low glass transition temperature film forming agents, and/or the alcohol component of the composition can “consist essentially of” secondary alcohol compounds.

For purposes of the present invention, the “basic and novel property” associated with compositions, components and methods which “consist essentially of” identified ingredients or actions is “attaching fake eye keratin materials to natural eye keratin materials.”

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Water

According to present invention, eye keratin material coating compositions comprising water are provided. The compositions of the present invention are not anhydrous.

Preferably, compositions of the present invention comprise from about 10% to about 75% water, preferably from about 20% to about 70% water, preferably from about 25% to about 60% water, and preferably from about 30% to about 55% water by weight with respect to the total weight of the composition, including all ranges and subranges therebetween.

### Low Glass Transition Temperature (Tg) Film Forming Agent

According to the present invention, compositions comprising at least one low glass transition temperature (Tg) film forming agent are provided. According to preferred embodiments, the at least one low glass transition temperature (Tg) film forming agent comprises at least one low glass transition temperature (Tg) film forming dispersion of film forming particles in aqueous phase. Such a dispersion of particles in aqueous phase is more generally known as latex.

As noted above, “Low Tg” or “Low Glass Transition Temperature” as used herein means a glass transition temperature (Tg) of −30° C. or lower, preferably −40° C. or lower, preferably −50° C. or lower, and preferably −60° C. or lower. According to preferred embodiments, low glass transition temperature film forming agents are film forming agents having a glass transition temperature from about −30° C. to about −100° C. preferably from about −40° C. to about −85° C., and preferably from about −45° C. to about −75° C., including all ranges and subranges therebetween.

Suitable polymers for the film-forming particles that may be used in the compositions of the present invention include, but are not limited to, synthetic polymers, free-radical type or polycondensate type polymers, and mixtures thereof.

Preferably, the polymers for the film-forming particles may be selected from vinyl (co)polymers, (meth)acrylic (co)polymers, urethane (co)polymers, and mixtures thereof. Advantageously, the polymer for the film-forming particles is selected from a styrene-(meth)acrylic and (meth)acrylic copolymer, a vinyl acetate and (meth)acrylic copolymer, and mixtures thereof.

Polymers for the film-forming particles of the free-radical type may be chosen, for example, from vinyl polymers or copolymers, such as acrylic polymers.

Vinyl film-forming polymers can result from the polymerization of monomers comprising at least one ethylenic unsaturation and at least one acidic group and/or esters of these acidic monomers and/or amides of these acidic monomers. Monomers comprising at least one acid group which may be used include, for example, α,β-ethylenic unsaturated carboxylic acids such as acrylic acid, methacrylic acid, crotonic acid, maleic acid or itaconic acid. (Meth)acrylic acid and crotonic acid are, for example, used. Preferably, (meth)acrylic acid is used.

The esters of acidic monomers can be chosen, for example, from (meth)acrylic acid esters (also known as (meth)acrylates), such as (meth)acrylates of an alkyl, for example, a C1-C30 alkyl, such as a C1-C20 alkyl, (meth)acrylates of an aryl, such as a C6-C10 aryl, and (meth)acrylates of a hydroxyalkyl, such as a C2-C6 hydroxyalkyl. Among the alkyl (meth)acrylates that may be mentioned, examples include methyl methacrylate, ethyl methacrylate, butyl methacrylate, isobutyl methacrylate, 2-ethylhexyl methacrylate, lauryl methacrylate and cyclohexyl methacrylate. Among the hydroxyalkyl (meth)acrylates that may be mentioned, examples include hydroxyethyl acrylate, 2-hydroxypropyl acrylate, hydroxyethyl methacrylate and 2-hydroxypropyl methacrylate. Among the aryl (meth)acrylates that may be mentioned, examples include benzyl acrylate and phenyl acrylate. The (meth)acrylic acid esters that may be used are, for example, alkyl (meth)acrylates.

The alkyl group of the esters may be substituted. For example, the alkyl group of the esters may be either fluorinated or perfluorinated, i.e., some or all of the hydrogen atoms of the alkyl group are substituted with fluorine atoms. Further, examples of amides of the acid monomers that may be mentioned include (meth)acrylamides, such as N-alkyl (meth)acrylamides, for example, of a C₂-C₁₂ alkyl. Among the N-alkyl (meth)acrylamides that may be mentioned, examples include N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or esters thereof and/or amides thereof, such as those mentioned above. Examples of vinyl esters that may be mentioned include vinyl acetate, ethylene vinyl acetate, vinyl neodecanoate, vinyl pivalate, vinyl benzoate and vinyl t-butylbenzoate. Styrene monomers that may be mentioned include styrene and α-methylstyrene.

Among the film-forming polycondensates that may be mentioned, examples include polyurethanes, polyesters, polyesteramides, polyamides, epoxyester resins and polyureas, and modifications or derivatives of any of these.

The polyurethanes may be chosen from anionic, cationic, nonionic or amphoteric polyurethanes, polyurethane-acrylics, polyurethane-polyvinylpyrrolidones, polyester-polyurethanes, polyether-polyurethanes, polyureas and polyurea-polyurethanes, and mixtures thereof.

The polyesters may be obtained, in a known manner, by polycondensation of dicarboxylic acids with polyols, such as diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalene-dicarboxylic acid and 2,6-naphthalenedicarboxylic acid. These dicarboxylic acid monomers may be used alone or as a combination of at least two dicarboxylic acid monomers. Among these monomers, phthalic acid, isophthalic acid and terephthalic acid may, for example, be used.

The diol may be chosen from aliphatic, alicyclic and aromatic diols. The diol used is, for example, chosen from ethylene glycol, diethylene glycol, triethylene glycol, 1,3-propanediol, cyclohexanedimethanol and 4-butanediol. Other polyols that may be used include glycerol, pentaerythritol, sorbitol and trimethylolpropane.

The polyesteramides may be obtained in a manner analogous to that of the polyesters, by polycondensation of diacids with diamines or amino alcohols. Diamines that may be used include, for example, ethylenediamine, hexamethylenediamine and meta- or para-phenylenediamine. An amino alcohol that may be used is, for example, monoethanolamine.

The polyester may also comprise at least one monomer bearing at least one —SO₃M group, wherein M is chosen from a hydrogen atom, an ammonium ion NH₄⁺ and a metal ion such as an Na⁺, Li⁺, K⁺, Mg²⁺, Ca²⁺, Cu²⁺, Fe²⁺ or Fe³⁺ ion. A difunctional aromatic monomer comprising such an —SO₃M group may, for example, be used.

The aromatic nucleus of the difunctional aromatic monomer also comprising an —SO₃M group as described above may be chosen, for example, from benzene, naphthalene, anthracene, biphenyl, oxybiphenyl, sulfonylbiphenyl and methylenebiphenyl nuclei. Among the difunctional aromatic monomers also comprising an —SO₃M group, mention may be made, for example, of sulfoisophthalic acid, sulfoterephthalic acid, sulfophthalic acid, 4-sulfonaphthalene-2,7-dicarboxylic acid.

The copolymers used are, for example, those based on isophthalate/sulfoisophthalate, such as copolymers obtained by condensation of diethylene glycol, cyclohexanedimethanol, isophthalic acid and sulfoisophthalic acid.

The polymer for the film forming particles may also be a liposoluble polymer. Examples of the liposoluble polymer that may be mentioned include copolymers of a vinyl ester (wherein the vinyl group is directly linked to the oxygen atom of the ester group and the vinyl ester comprises a radical chosen from saturated, linear or branched hydrocarbon-based radicals of 1 to 19 carbon atoms, linked to the carbonyl of the ester group) and of at least one other monomer, which may be a vinyl ester (different from the vinyl ester already present), an α-olefin (comprising from 8 to 28 carbon atoms), an alkyl vinyl ether (the alkyl group of which comprises from 2 to 18 carbon atoms) or an allylic or methallylic ester (comprising a radical chosen from saturated, linear or branched hydrocarbon-based radicals of 1 to 19 carbon atoms, linked to the carbonyl of the ester group).

These copolymers may be crosslinked using crosslinking agents that may be either of the vinylic type or of the allylic or methallylic type, such as tetraallyloxyethane, divinylbenzene, divinyl octanedioate, divinyl dodecanedioate and divinyl octadecanedioate.

Examples of these copolymers which may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Further examples of the liposoluble film-forming polymers include liposoluble copolymers, such as those resulting from the copolymerization of vinyl esters comprising from 9 to 22 carbon atoms or of alkyl acrylates or methacrylates, wherein the alkyl radicals comprise from 10 to 20 carbon atoms. Such liposoluble copolymers may be chosen, for example, from polyvinyl stearate, polyvinyl stearate crosslinked with the aid of divinylbenzene, of diallyl ether or of diallyl phthalate copolymers, polystearyl (meth)acrylate, polyvinyl laurate and polylauryl (meth)acrylate copolymers, it being possible for these poly(meth)acrylates to be crosslinked with the aid of ethylene glycol dimethacrylate or tetraethylene glycol dimethacrylate. The liposoluble copolymers described above are known and are described, for example, in French patent application FR-A-2 232 303; they may have a weight-average molecular weight ranging, for example, from 2,000 to 500,000 such as from 4,000 to 200,000.

Among the liposoluble film-forming polymers which may be used herein, mention may also be made, for example, of polyalkylenes such as copolymers of C₂-C₂₀ alkenes, such as polybutene, alkylcelluloses with a linear or branched, saturated or unsaturated C₁-C₈ alkyl radical, for instance ethylcellulose and propylcellulose, homopolymers or copolymers of vinylpyrrolidone (VP), such as copolymers of vinylpyrrolidone and of C₂-C₄₀ alkene such as C₃-C₂₀ alkene. In addition to PVP homopolymers, among the VP copolymers which may be used herein, mention may be made, for example, of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Specific examples of aqueous dispersions of film-forming particles which may be used are the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by the company Avecia-Neoresins, “Dow Latex 432®” by the company Dow Chemical, “Daitosol 5000 AD®” or “Daitosol 5000 SJ” by the company Daito Kasey Kogyo; the aqueous dispersions of polyurethane sold under the names “Neorez R-981®” and “Neorez R-974®” by the company Avecia-Neoresins, “Avalure UR-405®”, “Avalure UR-410®”, “Avalure UR-425®”, “Avalure UR-450®”, “Sancure 875®”, “Sancure 861®”, “Sancure 878®” and “Sancure 2060®” by the company Goodrich, “Impranil 85®” by the company Bayer and “Aquamere H-151®” by the company Hydromer; vinyl dispersions, for instance “Mexomer PAM” and also acrylic dispersions in isododecane, for instance “Mexomer PAP” by the company Chimex.

Further specific examples of latex polymers for use in the present invention further include ethylhexyl acrylate/hema copolymer (and) acrylates/diethylaminoethyl methacrylate/ethylhexyl acrylate copolymer (Syntran®PC 5775), styrene/acrylates/ammonium methacrylate copolymer (Syntran®5760, Syntran®5009, Syntran®PC5620), polyacrylate-21 (and) acrylates/dimethylaminoethyl methacrylate copolymer (Syntran®PC5100, Syntran®PC5776, Eudragit®E 100, Jurymer ET-410C), styrene/acrylates/ammonium methacrylate copolymer (Syntran®5009 CG), olefin/acrylate grafted polymer (and) sodium laureth sulfate (and C12-15 SEC-pareth 15 (Syntran®EX108), acrylates copolymer (Aculyn®33A Polymer, Avalure®Ace 210/120/315 Acrylic Copolymer, Carbopol® Aqua SF-1 Polymer, Coatex®Co 633, Eliclear®380/700/4U, Eudragit® L 100, Joncryl®85, Luviflex®Soft), acrylates/ethylhexyl acrylate copolymer. The Syntran® polymers are commercially available from the supplier Interpolymer Corp.

Further specific examples include an ethyl acrylates/methyl methacrylates copolymer emulsion (chemical name) (INCI name: water (and) acrylates copolymer), which is commercially available from Kobo Products, Inc, (South Plainfield, N.J.) and Daito Kasei Kogyo Co., Ltd., under the trade name Daitosol AD (which contains water, ethyl acrylates/methyl methacrylates copolymer, sodium dehydroacetate, and Laureth-20 (lauryl alcohol and oxirane); an ethyl methacrylates/N-butyl acrylates/2-methylhexyl acrylates copolymer emulsion (chemical name) (INCI name: water (and) acrylates/ethylhexyl acrylates copolymer), which is also commercially available from Kobo Products, Inc. and Daito Kasei Kogyo Co., Ltd., under the trade name Daitosol SJ (which contains water, ethyl methacrylates/N-butyl acrylates/2-methylhexyl acrylates copolymer, and Laureth-20); an alkyl(meth)acrylates copolymer emulsion (INCI name: acrylates copolymer), which is commercially available from Nippon LSC Ltd., under the trade name Yodosol GH34F; and an acrylates/ammonium methacrylates copolymer (INCI name) (CAS No. 25212-88-8), commercially available from Ganz Chemical under the tradename ULTRASOL (which also contains water, zinc oxide, sodium lauryl sulfate, and methylparaben).

Preferably, the low glass transition temperature film-forming agent(s) is/are present in the compositions of the present invention in amounts of active material (e.g., solid content) generally ranging from about 5% to about 60%, preferably from about 10% to about 55%, preferably from about 15% to about 30%, and preferably from about 20% to about 25%, by weight, based on the total weight of the composition, including all ranges and subranges in between, such as, for example 25% to 60%, 30% to 55%, 8% to 18%, 2% to 10%, 10% to 25%, 12.5% to 30%, etc.

### Secondary Alcohol Compound

According to the present invention, compositions comprising at least one secondary alcohol compound are provided.

“Secondary alcohol compound” as used herein refers to a compound having at least one secondary alcohol group. “Secondary alcohol group” has its generally accepted meaning: the carbon atom to which the alcohol group is attached in a compound has only one hydrogen attached. Specific examples of secondary alcohol compounds include, but are not limited to, compounds containing a single secondary alcohol group such as glycerin, 2-propanol, 2-butanol, 2-pentanol, 3-pentanol, etc., compounds containing two secondary alcohol groups such 2,3-butanediol, 2,3-pentanediol, etc., glycols such as propylene glycol, butylene glycol, pentylene glycol, hexylene glycol, etc., and compounds containing three or more secondary alcohol groups such as sorbitol, xylitol, erythritol, mannitol, etc.

According to the present invention, the secondary alcohol compound or mixture of secondary alcohol compounds present in the composition has (if only one secondary alcohol-containing compound is present) or collectively have (if two or more secondary alcohol-containing compounds are present) molecular weight(s) such that the weighted average ratio of molecular weight/number of secondary alcohol groups for all secondary alcohol compounds present is less than 69, preferably 67 or less, preferably 64 or less, preferably 61 or less, preferably 60 or less, preferably 55 or less, preferably 50 or less, preferably 48 or less, and preferably 45 or less.

“Weighted average ratio of molecular weight/number of secondary alcohols groups” is calculated as follows, using sorbitol alone, propylene glycol alone, and sorbitol and propylene glycol present in a composition in a 1:1 weight ratio for demonstrative purposes:

Sorbitol: molecular weight=182; number of secondary alcohol groups=4. Ratio of molecular weight/number of secondary alcohols groups for sorbitol alone=45.5

Propylene glycol: molecular weight=76; number of secondary alcohol groups=1. Ratio of molecular weight/number of secondary alcohols groups for propylene glycol alone=76.

For a 1:1 weight ratio of sorbitol and propylene glycol present in a composition, “weighted average ratio of molecular weight/number of secondary alcohols groups”=[(45.5×1)+(76×1))/(1+1)=60.75.

Alternatively, and by way of example, for a 4:1 weight ratio of sorbitol and butylene glycol (which has a molecular weight of 90 and 1 secondary alcohol group), “weighted average ratio of molecular weight/number of secondary alcohols groups”=[(45.5×4)+(90×1)]/(4+1)=54.4.

Preferably, the at least one secondary alcohol compound is/are present in the compositions of the present invention in amounts ranging from about 0.5% to about 25%, preferably from about 1% to about 20%, preferably from about 2.5% to about 15%, preferably from about 3% to about 10%, and preferably from about 4% to about 8%, by weight, based on the total weight of the composition, including all ranges and subranges in between, such as, for example 0.5% to 8%, 1% to 5%, 8% to 18%, 2% to 10%, 1% to 7.5%, 2.5% to 8%, less than 15%, less than 10%, less than 5%, etc.

### Nonionic Amphiphilic Thickening Agents

According to preferred embodiments of the present invention, compositions optionally further comprising at least one nonionic amphiphilic thickening agent are provided. By “nonionic amphiphilic thickening agent,” it is meant that the thickening agent has at least one hydrophilic portion and at least one hydrophobic portion, and the thickening agent is nonionic. Preferably, the at least one nonionic amphiphilic thickening agent additionally possesses film forming properties in addition to aqueous phase thickening properties.

Suitable examples of hydrophilic and/or hydrophobic monomers for inclusion into the at least one nonionic amphiphilic thickening agents include, but are not limited to, vinyl alcohol (VA), vinylpyrrolidone (VP), C₂ to C₃₀ alkene (such as, for example, C₃ to C₂₂. alkene), acrylic acid, C₂ to C₃₀ acrylates, polyethylene glycol (PEG) or polypropylene glycol (PPG) groups, etc.

Preferably, the at least one nonionic amphiphilic thickening agent is a vinylpyrrolidone (VP) copolymer, preferably containing at least one C₂ to C₃₀ alkene monomer. As specific non-limiting examples of VP copolymers suitable for use in the present compositions, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene, VP/methacrylamide/vinyl imidazole copolymer, and VP/acrylic acid/lauryl methacrylate copolymer.

According to some embodiments, however, the nonionic amphiphilic thickening component in the compositions of the present invention does not include a thickening agent which is a copolymer including VP and imidazole monomers, in particular VP/methacrylamide/vinyl imidazole copolymer.

Preferably, if present, the at least one nonionic amphiphilic thickening agent is present in the compositions of the present invention in amounts of active material (e.g., solid content) generally ranging from about 0.5% to about 20%, preferably from about 1% to about 15%, preferably from about 2.5% to about 10%, and preferably from about 4% to about 8%, by weight, based on the total weight of the composition, including all ranges and subranges in between, such as, for example 0.1% to 15%, 0.1% to 5%, 8% to 18%, 2% to 10%, 1% to 5%, 2.5% to 8%, less than 15%, less than 10%, less than 5%, less than 3%, less than 2%, less than 1%, etc.

Preferably, nonionic amphiphilic thickening agent(s) and low glass transition temperature film forming agent(s) are present in the compositions of the present invention in weight ratios (active material) of nonionic amphiphilic thickening agent to low glass transition temperature film forming agent of from about 1:250 to about 1:1, preferably from about 1:150 to about 1:20, and preferably from about 1:100 to about 1:50, including all ranges and subranges therebetween, such as, for example 1:200 to 1:25. 1:175 to 1:60, etc.

According to preferred embodiments, compositions of the present invention are substantially free of, devoid of, or free of hydrophilic thickening agent(s).

According to preferred embodiments, thickening agent components of the compositions of the present invention consist essentially of, or consist of, nonionic amphiphilic thickening agent(s).

### Water-Soluble Agent

According to preferred embodiments of the present invention, compositions optionally further comprising at least one water-soluble agent selected from the group consisting of C1-C5 monoalcohols, hydrotropic agent(s), and mixtures thereof, are provided.

Suitable examples of C1-C5 monoalcohols include compounds having a C1-C5 alkane chain, and a single hydroxyl function (OH) such as, for example, methanol, ethanol, propanol, isopropanol, butanol, and mixtures thereof;

Suitable examples of hydrotropic agent(s) include hydrotropic agent(s) selected from the group consisting of salt(s) of aromatic acid(s). Salt(s) of aromatic acid(s) include compounds such as, for example, salts such as sodium or potassium salts of compounds comprising an aromatic structure (for example, benzyl, furanyl, thiophenyl, pyrrolyl, etc.) and at least one carboxyl group substituent on the aromatic structure (e.g., sodium benzoate). Optionally, the aromatic structure may contain at least one substituent other than the at least one carboxyl group such as, for example, lower alkyl (C1-C3), hydroxyl, and/or halogen. For example, a hydroxyl-substituted sodium salt of benzoic acid (also known as sodium salicylate) is a suitable example.

Preferably, if present, the at least one water-soluble agent selected from the group consisting C1-C5 monoalcohols, hydrotropic agent(s), and mixtures thereof, is/are present in the compositions of the present invention in amounts ranging from about 1% to about 25%, preferably from about 2% to about 20%, preferably from about 2.5% to about 15%, preferably from about 3% to about 10%, and preferably from about 4% to about 8%, by weight, based on the total weight of the composition, including all ranges and subranges in between, such as, for example 5% to 10%, 8% to 18%, 2% to 10%, 1% to 7.5%, 2.5% to 8%, etc.

According to preferred embodiments, if present, the at least one water-soluble agent selected from the group consisting of C₁-C₅ monoalcohols, hydrotropic agent(s), and mixtures thereof, is/are present in the compositions of the present invention in amounts less than or equal to about 10% by weight, preferably less than or equal to 8% by weight, and preferably less than or equal to 7.5% by weight with respect to the total weight of the composition.

### Oil Phase

According to embodiments of the present invention, the compositions of the present invention may optionally further comprise at least one oil. “Oil” means any non-aqueous medium which is liquid at ambient temperature (25° C.) and atmospheric pressure (760 mm Hg). Suitable oils can be volatile or non-volatile.

Suitable oils include volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Suitable oils include non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Suitable oils include synthetic oils or esters of formula R₅COOR₆ in which R₅ represents a linear or branched higher fatty acid residue containing from 1 to 40 carbon atoms, including from 7 to 19 carbon atoms, and R6 represents a branched hydrocarbon-based chain containing from 1 to 40 carbon atoms, including from 3 to 20 carbon atoms, with R₆+R7³10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; pentaerythritol esters; and synthetic ethers containing from 10 to 40 carbon atoms.

If present, the oil(s) is/are present in the compositions of the present invention in an amount ranging from about 0.1% to about 20% by weight, more preferably from about 0.4% to about 15% by weight, and preferably from about 0.5% to about 10% by weight, based on the total weight of the composition, including all ranges and subranges within these ranges.

According to preferred embodiments, however, compositions of the present invention are substantially free of, devoid of, or free of oils.

According to preferred embodiments, compositions of the present invention are substantially free of, devoid of, or free of volatile oils such as, for example, isododecane and cyclomethicone.

According to preferred embodiments, compositions of the present invention are substantially free of, devoid of, or free of silicone oils such as, for example, dimethicone and cyclomethicone.

According to preferred embodiments, the oil component of the compositions of the present invention consists of non-volatile oils.

According to preferred embodiments, the oil component of the compositions of the present invention consists of hydrocarbon oils.

### Coloring Agents

According to embodiments of the present invention, compositions optionally further comprising at least one coloring agent are provided. Preferably, such colored compositions can be cosmetic compositions for making up eyelashes such as mascaras.

According to this embodiment, the at least one coloring agent is preferably chosen from pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, may be present in the composition in a concentration ranging up to 50% by weight of the total weight of the composition, such as from 0.1% to 20%, preferably from 0.1% to 15%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the coloring agent(s) may be present in the composition in a concentration ranging up to 25% by weight of the total weight of the composition, such as from 2.5% to 20%, and further such as from 5% to 15%, including all ranges and subranges therebetween.

However, it is possible for the compositions of the present invention to be free, substantially free, or devoid of coloring agent(s).

### Additional Additives

The composition of the invention can also comprise any additive usually used in the field under consideration. For example, dispersants such as poly(12-hydroxystearic acid), surfactants, antioxidants, essential oils, sunscreens, preserving agents, fragrances, fillers, neutralizing agents, cosmetic and dermatological active agents such as, for example, emollients, moisturizers, vitamins, essential fatty acids, silicone elastomers, pasty compounds, and mixtures thereof can be added. Compositions of the present invention can be free of, substantially free of, or devoid of (as defined above) of any of the ingredients discussed in this paragraph.

A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eye keratin materials of human beings.

The eye keratin material coating compositions of the present invention preferably possess sufficient tack and sufficient quick-drying (for example, 1 minute or less) properties such that, after the compositions are applied to eye keratin materials, false or artificial eye keratin materials can be adhered to the eye keratin materials by, through or using the applied eye keratin material coating compositions of the present invention by placing the false or artificial eye keratin materials on the applied composition and allowing the eye keratin material coating composition to dry. Further, the eye keratin material coating compositions can be applied to false (artificial) eye keratin materials or natural (real) eye keratin materials, if desired, to care for or make up the eye keratin materials and, as such, the eye keratin material coating compositions of the present invention can be multi-purpose.

According to preferred embodiments of the present invention, methods of applying fake (artificial) eye keratin materials such as artificial eyelashes or eyebrows to real (natural) eye keratin materials such as eyelashes, eyebrows or skin associated with eyes such as eyelids or brows comprising applying compositions of the present invention to natural eye keratin materials, applying fake eye keratin materials to the natural eye keratin materials upon which compositions of the present invention have been previously applied, and allowing the compositions of the present invention to dry so that the fake eye keratin materials attach to the natural eye keratin materials are provided.

According to preferred embodiments of the present invention, methods of applying fake (artificial) eye keratin materials such as artificial eyelashes or eyebrows to real (natural) eye keratin materials such as eyelashes, eyebrows or skin associated with eyes such as eyelids or brows comprising applying compositions of the present invention to fake eye keratin materials, applying fake eye keratin materials to the natural eye keratin materials, and allowing the compositions of the present invention to dry so that the fake eye keratin materials attach to the natural eye keratin materials are provided.

In accordance with the preceding preferred embodiments, the compositions of the present invention are applied topically to the desired area of the eye keratin materials (real and/or fake) in an amount sufficient to attach false (artificial) eye keratin materials to natural eye keratin materials. The compositions may be applied to the desired area as needed, preferably once or twice prior to attachment of the artificial eye keratin materials, and then allowed to dry after contacting the applied composition with artificial eye keratin materials for attachment to eye keratin materials. Preferably, the artificial eye keratin materials are contacted with the applied composition less than 1 minute after the composition has been applied to natural and/or fake eye keratin materials, more preferably less than 45 seconds after application.

Typically, artificial eye keratin materials attached to natural eye keratin materials do not need special removal techniques, and can be removed by gently pulling them away from eye keratin materials. However, typical removal methods and solutions can be used to remove the artificial eye keratin materials from natural eye keratin materials, if desired.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example I—Sample Formulations

| Ingredient | Preferred Range | Specific Range |
|---|---|---|
| Low Tg Film Former | 25-60% | 30-55% |
| Secondary Alcohol | 0.5-8% | 1-5% |
| Compound | | |
| Water | 10-75% | 30-55% |
| Nonionic amphiphilic | 0-15% | 0.1-5% |
| thickening agent | | |
| Water-Soluble Agent | 0-20% | 5-10% |
| Other (preservative, | 0-10% | 0-2% |
| coloring agent, etc.) | | |

### Example II—Composition Preparation

The compositions of the present invention can be prepared in a customary manner for eyelash products. For example, compositions in example III (bulk, 30 g) were prepared by speedmixing at 2000 RPM, 1 minute, using Hauschild SpeedMixer DAC 400.1 FVZ.

### Example III—Testing

### A. Testing Protocols

### 1. Tack

Tack is an indicator of initial attachment properties of a composition. Tack was evaluated on a continuum of 1 to 5 by evaluating sample ability to attach when contacting human skin, with representative points on the continuum being 1=low tack (not sticky), 3=some tack (some resistance removing finger from film surface), and 5=high tack (sticky, finger difficult to remove from film surface).

To evaluate tack, 1 g of product was weighed in a weight boat and allowed to air dry overnight (24 hours) in ambient conditions or in a fume hood to prepare each film. After drying, a finger was pressed on each film and then pulled in an upward direction, and the force needed to separate the finger from the film was gauged on the 1-5 continuum noted above.

### 2. Adhesion

Adhesion is an indicator of wear properties of a composition. Adhesion was evaluated on a continuum of 1 to 5 by evaluating sample ability to be removed from a container, with representative points on the continuum being 1=low adhesion (easy to remove), 3=some adhesion (some resistance to removal), and 5=high adhesion (difficult to remove).

To evaluate adhesion, approximately 1 g of each composition was poured into a polystyrene weigh boat and allowed to dry overnight in ambient conditions or in a fume hood. The adhesion of the dried composition to the weigh boat was determined by pulling film away from weigh boat, gauging the force needed to remove film from weigh boat surface.

### 3. Cohesion

Cohesion is an indicator of resistance properties of a composition. Cohesion was evaluated on a continuum of 1 to 5 by evaluating sample ability to rip upon stretching, with representative points on the continuum being was evaluated on a scale of 1 to 5, with 1=low cohesion (easy to rip film upon stretching), 3=some cohesion (some film ripping upon stretching), and 5=high cohesion (no rip of film upon stretching).

To evaluate cohesion, approximately 1 g of each composition was poured into a weigh boat and allowed to dry overnight in ambient conditions or in a fume hood. The cohesion of the dried composition was determined by removing a segment of film from the weigh boat, and gauging the force needed to create a rupture or tear in that segment.

### B. Experimental Testing

The following simplex formulations were prepared using a Hauschild SpeedMixer DAC 400.1 FVZ at 2000 RPM for 1 minute:

| Trial | Example | Specific Example |
|---|---|---|
| 1 | Amphiphilic thickener + | VP/VA copolymer (0.5%) + |
| | low Tg polymer | acrylates/ethylhexyl acrylate copolymer (99.5%) |
| | | White with blue hue, opaque viscous liquid. |
| 5 | Amphiphilic thickener + | VP/VA copolymer (0.5%) + |
| | low Tg polymer + aq. | acrylates/ethylhexyl acrylate copolymer (94.5%) + |
| | organic solvent | glycerin (5%) |
| | | White with blue hue, opaque viscous liquid (<#1) |
| 6 | Amphiphilic thickener + | VP/VA copolymer (0.5%) + |
| | low Tg polymer + aq. | acrylates/ethylhexyl acrylate copolymer (89.5%) + |
| | organic solvent | glycerin (10%) |
| | | White with blue hue, opaque viscous liquid (<<#1) |
| 11 | Low Tg polymer | Acrylates/ethylhexyl acrylate copolymer (100%) |
| | | White with blue hue, opaque viscous liquid (≤#1) |
| 22 | Amphiphilic thickener + | VP/VA copolymer (0.5%) + |
| | low Tg polymer + aq. | acrylates/ethylhexyl acrylate copolymer (79.5%) + |
| | organic solvent | glycerin (20%) |
| | | White with blue hue, opaque liquid. |
| 23 | Amphiphilic thickener + | VP/VA copolymer (0.5%) + |
| | low Tg polymer + aq. | acrylates/ethylhexyl acrylate copolymer (94.5%) + |
| | organic solvent | propylene glycol (5%) |
| | | White with blue hue, opaque viscous liquid |
| 24 | Amphiphilic thickener + | VP/VA copolymer (0.5%) + |
| | low Tg polymer + aq. | acrylates/ethylhexyl acrylate copolymer (89.5%) + |
| | organic solvent | propylene glycol (10%) |
| | | White with blue hue, opaque liquid. |
| 25 | Amphiphilic thickener + | VP/VA copolymer (0.5%) + |
| | low Tg polymer + aq. | acrylates/ethylhexyl acrylate copolymer (79.5%) + |
| | organic solvent | propylene glycol (20%) |
| | | White with slight blue hue, opaque liquid. Not stable. |
| 26 | Amphiphilic thickener + | VP/VA copolymer (0.5%) + |
| | low Tg polymer + aq. | acrylates/ethylhexyl acrylate copolymer (94.5%) + |
| | organic solvent | hexylene glycol (5%) |
| | | White with blue hue, opaque viscous liquid |
| 27 | Amphiphilic thickener + | VP/VA copolymer (0.5%) + |
| | low Tg polymer + aq. | acrylates/ethylhexyl acrylate copolymer (89.5%) + |
| | organic solvent | hexylene glycol (10%) |
| | | White with blue hue, opaque liquid. Separates in film |
| | | (clear + white liquid). |
| 28 | Amphiphilic thickener + | VP/VA copolymer (0.5%) + |
| | low Tg polymer + aq. | acrylates/ethylhexyl acrylate copolymer (79.5%) + |
| | organic solvent | hexylene glycol (20%) |
| | | White with slight blue hue, opaque liquid. Clear gel |
| | | bodies. Not stable. |
| 29 | Amphiphilic thickener + | VP/VA copolymer (0.5%) + |
| | low Tg polymer + aq. | acrylates/ethylhexyl acrylate copolymer (94.5%) + |
| | organic solvent | sorbitol (5%) |
| | | White with blue hue, opaque viscous liquid |
| 30 | Amphiphilic thickener + | VP/VA copolymer (0.5%) + |
| | low Tg polymer + aq. | acrylates/ethylhexyl acrylate copolymer (89.5%) + |
| | organic solvent | sorbitol (10%) |
| | | White with blue hue, opaque liquid. |
| 31 | Amphiphilic thickener + | VP/VA copolymer (0.5%) + |
| | low Tg polymer + aq. | acrylates/ethylhexyl acrylate copolymer (79.5%) + |
| | organic solvent | sorbitol (20%) |
| | | White with slight blue hue, opaque liquid. |

*acrylates/ethylhexyl acrylate copolymer is 55% active material; VP/VA copolymer is 50% active material.

| Trial | Example | Specific Example |
|---|---|---|
| 41 | Amphiphilic thickener + | VP/VA copolymer (0.5%) + |
| | low Tg polymer + aq. | acrylates/ethylhexyl acrylate copolymer (94.5%) + |
| | organic solvent 1 | glycerin (2.5%) + |
| | (2.5%) + aq. organic | sorbitol (2.5%) |
| | solvent 2 (2.5%) | White with blue hue, opaque viscous liquid (slt. <#1). |
| | 5%, 1:1 ratio | |
| 42 | Amphiphilic thickener + | VP/VA copolymer (0.5%) + |
| | low Tg polymer + aq. | acrylates/ethylhexyl acrylate copolymer (94.5%) + |
| | organic solvent 1 | glycerin (2.5%) + |
| | (2.5%) + aq. organic | propylene glycol (2.5%) |
| | solvent 2 (2.5%) | White with blue hue, opaque viscous liquid (slt. <#1). |
| | 5%, 1:1 ratio | |
| 43 | Amphiphilic thickener + | VP/VA copolymer (0.5%) + |
| | low Tg polymer + | acrylates/ethylhexyl acrylate copolymer (94.5%) + |
| | aq. organic solvent 2 | propylene glycol (2.5%) + |
| | (2.5%) + aq. organic | sorbitol (2.5%) |
| | solvent 7 (2.5%) | White with blue hue, opaque viscous liquid (slt. <#1). |
| | 5%, 1:1 ratio | |
| 44 | Amphiphilic thickener + | VP/VA copolymer (0.5%) + |
| | low Tg polymer + aq. | acrylates/ethylhexyl acrylate copolymer (94.5%) + |
| | organic solvent 1 | glycerin (2.5%) + |
| | (2.5%) + aq. organic | denatured alcohol (2.5%) |
| | solvent 2A (2.5%) | White with blue hue, opaque viscous liquid (slt. <#1). |
| | 5%, 1:1 ratio | |

* acrylates/ethylhexyl acrylate copolymer is 55% act. VP/VA copolymer VP/VA copolymer is 50% act.

These compositions were tested in triplicate for tack, adhesion and cohesion properties as described above. The results for each property for each composition for each test is set forth below:

Level of Tack (films):

| Trial | N = 1 | N = 2 | N = 3 | |
|---|---|---|---|---|
| 1 | 5 | 4 | 4 | 4.333 |
| 5 | 5 | 5 | 5 | 5 |
| 6 | 3 | 1 | 2 | 2 |
| 11 | 5 | 5 | 5 | 5 |
| 22 | 2 | 1 | 1 | 1.333 |
| 23 | 5 | 4 | 5 | 4.667 |
| 24 | 5 | 4 | 4 | 4.333 |
| 26 | 4 | 3 | 4 | 3.667 |
| 27 | 3 | 1 | 2 | 2 |
| 29 | 4 | 5 | 4 | 4.333 |
| 30 | 3 | 4 | 3 | 3.333 |
| 31 | 2 | 2 | 2 | 2 |
| 41 | 5 | 5 | 5 | 5 |
| 42 | 5 | 5 | 5 | 5 |
| 43 | 4 | 5 | 5 | 4.667 |
| 44 | 5 | 5 | 5 | 5 |

Level of Adhesion (films)

| Trial | N = 1 | N = 2 | N = 3 | |
|---|---|---|---|---|
| 1 | 4 | 4 | 3 | 3.667 |
| 5 | 1 | 1 | 4 | 2 |
| 6 | 1 | 1 | 1 | 1 |
| 11 | 4 | 4 | 4 | 4 |
| 22 | 1 | 1 | 1 | 1 |
| 23 | 4 | 4 | 3 | 3.667 |
| 24 | 3 | 4 | 3 | 3.333 |
| 26 | 2 | 4 | 4 | 3.333 |
| 27 | 2 | 2 | 2 | 2 |
| 29 | 3 | 3 | 3 | 3 |
| 30 | 2 | 2 | 2 | 2 |
| 31 | 1 | 2 | 3 | 2 |
| 41 | 1 | 3 | 2 | 2 |
| 42 | 3 | 3 | 4 | 3.333 |
| 43 | 2 | 3 | 4 | 3 |
| 44 | 1 | 3 | 2 | 2 |

Level of Cohesion (films)

| Trial | N = 1 | N = 2 | N = 3 | |
|---|---|---|---|---|
| 1 | 4 | 2 | 3 | 3 |
| 5 | 5 | 4 | 4 | 4.333 |
| 6 | 5 | 1 | 3 | 3 |
| 11 | 4 | 1 | 3 | 2.667 |
| 22 | 1 | 1 | 1 | 1 |
| 23 | 2 | 2 | 3 | 2.333 |
| 24 | 1 | 3 | 3 | 2.333 |
| 26 | 2 | 3 | 3 | 2.667 |
| 27 | 1 | 1 | 2 | 1.333 |
| 29 | 4 | 3 | 4 | 3.667 |
| 30 | 4 | 4 | 3 | 3.667 |
| 31 | 2 | 2 | 2 | 2 |
| 41 | 4 | 4 | 4 | 4 |
| 42 | 2 | 2 | 3 | 2.333 |
| 43 | 3 | 3 | 4 | 3.333 |
| 44 | 4 | 3 | 3 | 3.333 |

Trials 29 and 43 exhibited superior tack, adhesion and cohesion properties, obtaining 3 or higher for all properties tested, demonstrating unexpected beneficial results associated with lower amounts of compounds containing secondary alcohol groups where the weighted average ratio of molecular weight/number of secondary alcohol groups for the at least one secondary alcohol compound present in the composition was less than 69.

Set forth below is a summary of testing performed for various alcohols, including non-secondary alcohols, where ** indicates the composition obtaining 3 or higher for all properties tested.

| | # secondary | | MW/#sec alcohol |
|---|---|---|---|
| | alcohol | | groups (weighted |
| | groups | MW | average for blends) |
| Sorbitol** | 4 | 182 | 45 |
| Sorbitol + PG (1:1)** | 4, 1 | | (45 + 76)/2 = 61 |
| PG | 1 | 76 | 76 |
| Sorbitol + Glycerin (1:1) | 4, 1 | | (45 + 92)/2 = 69 |
| PG + Glycerin (1:1) | 1, 1 | 76, 92 | (76 + 92)/2 = 89 |
| Glycerin | 1 | 92 | 92 |
| Hexylene glycol | 0 | 118 | — |
| Phenoxyethanol | 0 | 138 | — |
| Benzyl alcohol | 0 | 108 | — |
| Menthol | 1 | 157 | 157 |
| Butylene Glycol | 1 | 90 | 90 |
| Ethylhexyl glycerin | 1 | 204 | 204 |

What is claimed is:

1. An eye keratin material coating composition comprising water, at least one low glass transition temperature (Tg) film-forming agent, and at least one secondary alcohol compound, wherein a weighted average ratio of molecular weight/number of secondary alcohol groups for the at least one secondary alcohol compound present in the composition less than 69.

2. The composition of claim 1, further comprising at least one water-soluble agent selected from the group consisting of C1-C5 monoalcohols, salt(s) of aromatic acid(s), and mixtures thereof.

3. The composition of claim 2, wherein at least one salt of an aromatic acid is present in the composition and is selected from the group consisting of sodium benzoate, sodium salicylate, and mixtures thereof.

4. The composition of claim 2, wherein the at least one water-soluble agent is present in an amount of about 10% or less by weight with respect to the weight of the total composition.

5. The composition of claim 1, further comprising at least one nonionic amphiphilic thickening agent.

6. The composition of claim 1, wherein the composition is substantially free of coloring agent.

7. The composition of claim 1, wherein the composition is substantially free of hydrophilic thickening agent.

8. The composition of claim 1, wherein the composition is substantially free of oil and wax.

9. The composition of claim 1, wherein the composition is substantially free of surfactant.

10. A method of applying artificial eye keratin material to natural eye keratin material comprising applying an eye keratin material coating composition comprising water, at least one low glass transition temperature (Tg) film-forming agent, at least one secondary alcohol compound, wherein a weighted average ratio of molecular weight/number of secondary alcohol groups for the at least one secondary alcohol compound present in the composition less than 69, to natural eye keratin material, applying artificial eye keratin material to the natural eye keratin material upon which the composition has been applied, and allowing the composition to dry so that the artificial eye keratin material attaches to the natural eye keratin material.

11. The method of claim 10, wherein the natural eye keratin material is an eyelid or a brow.

12. The method of claim 10 wherein the natural eye keratin material is an eyelash or eyebrow.

13. The method of claim 10, wherein the artificial eye keratin material comprises fibers attached to a substrate.
